(19) United States
(12) Patent Application Publication
(10) Pub. No.: US 2023/0233182 A1
(43) Pub. Date: Jul. 27, 2023

(54) SELF-DISINFECTING ULTRASOUND PROBE

(71) Applicant: Becton, Dickinson and Company, Franklin Lakes, NJ (US)

(72) Inventors: Christopher Quach, Salt Lake City, UT (US); James Freasier, Salt Lake City, UT (US); Brendan Laine, Draper, UT (US)

(21) Appl. No.: 18/076,222

(22) Filed: Dec. 6, 2022

Related U.S. Application Data
(60) Provisional application No. 63/302,930, filed on Jan. 25, 2022.

(51) Int. Cl. A61B 8/00; G01S 7/52; A01N 25/10; A01N 59/16; A01P 1/00

(52) U.S. Cl. CPC A61B 8/4444; G01S 7/52079; A01N 25/10; A01N 59/16; A01P 1/00

(57) ABSTRACT

An ultrasound probe with antimicrobial surface activity includes a housing, wherein the housing has an antimicrobial compound present at an exterior surface of the probe housing in sufficient concentration to provide antimicrobial activity with microbes that contact the exterior surface of the ultrasound probe. A plurality of antimicrobial compounds may be present at the exterior surface of the probe housing in sufficient concentration to provide antimicrobial activity. The antimicrobial compound may be an additive to a polymeric material from which the probe housing is manufactured. The antimicrobial compound may be present in an antimicrobial coating disposed on the exterior surface of the probe housing. The antimicrobial activity may provide log reductions in gram+ and gram− bacteria after 24 hours in the range from 3 to 7.

### CROSS-REFERENCE TO RELATED APPLICATIONS

This application claims the benefit of U.S. Provisional Application No. 63/302,930, filed Jan. 25, 2022, and entitled SELF-DISINFECTING ULTRASOUND PROBE, which is incorporated herein in its entirety.

### BACKGROUND

The disclosed invention relates to a novel ultrasound probe with enhanced antimicrobial efficacy. An exterior surface of the probe may be impregnated and/or coated with one or more antimicrobial compounds to provide surface antimicrobial functionality. The disclosed ultrasound probe provides enhanced antimicrobial efficacy without significant disruption of a clinician's workflow. The disclosed ultrasound probe may eliminate the need for high level disinfection (HLD) instruments such as ultraviolet (UV) chambers or hydrogen peroxide washes.

Ultrasound probes are used in a variety of medical procedures to produce an image of structures within the body. For example, ultrasound probes are commonly used to monitor, diagnose, and evaluate different internal organs, structures, and conditions. Ultrasound probes are also used in certain percutaneous needle and catheter placement procedures. Most ultrasound procedures are done using an ultrasound probe outside the body, though some involve placing a probe device within the body.

Ultrasound probes must be properly disinfected following a medical procedure. Current disinfection methods for ultrasound probes rely on the use of capital equipment. These disinfection methods utilize technologies such as hydrogen peroxide-based washing systems or ultraviolet disinfection. One known hydrogen peroxide washing system for ultrasound probes is provided by GE Healthcare under the tradename Trophon. One known UV chamber for disinfecting ultrasound probes is provided by Germitec under the tradename Chronos. Such systems require additional training for clinicians and impact the clinician's workflow negatively by adding extra steps to it. They also require purchase of expensive capital equipment.

The disclosed self-disinfecting ultrasound probe may eliminate the need for purchase of capital equipment, save time for the clinician, and simplify the maintenance of the ultrasound probe. The disclosed technology also disinfects passively without disrupting the clinician's workflow and current practices.

The self-disinfecting ultrasound probe disclosed herein is not limited to embodiments that solve any disadvantages or that operate only in environments such as those described above. Rather, this background is only provided to illustrate one example technology area where some implementations described herein may be practiced.

### SUMMARY

The present disclosure relates generally to ultrasound probes having self-disinfecting properties to provide enhanced antimicrobial efficacy. In particular, the present disclosure relates to the use of antimicrobial compounds to passively disinfect the surface of an ultrasound probe to provide the disinfection. The disclosed antimicrobial compounds provide the ultrasound probe surface with antimicrobial efficacy and reduce the risk of infection for patients who may contact the ultrasound probe. In some embodiments, one or more antimicrobial compound additives may be incorporated into the probe housing. In some embodiments, one or more antimicrobial compounds may be incorporated into a coating on the surface of the ultrasound probe. In some embodiments, a combination of one or more antimicrobial compound additives and one or more antimicrobial compounds incorporated into a coating on the surface of the ultrasound probe may be utilized to provide self-disinfecting properties.

In some embodiments an ultrasound probe with antimicrobial surface activity comprises a housing, wherein the housing comprises an antimicrobial compound. The antimicrobial compound is present at an exterior surface of the housing in sufficient concentration to provide antimicrobial activity with microbes that contact the exterior surface of the ultrasound probe.

In some embodiments the antimicrobial compound includes but is not limited to an antimicrobial compound selected from hydrogen peroxide, zinc oxide, ionic zinc (Zn²⁺), chlorhexidine, copper, silver (ionic), silver nanoparticles, triclosan, polyhexamethylene biguanide (PHMB), quaternary ammonium compounds, guanidine, guanidinium compounds, porphyrin, methylene blue, toluidine blue, and rose bengal.

In some embodiments the housing comprises a plurality of antimicrobial compounds. A plurality of antimicrobial compounds may provide a broader spectrum of antimicrobial activity.

In some embodiments the antimicrobial compound is ionic zinc (Zn²⁺).

In some embodiments the antimicrobial compound is selected from zinc oxide (ZnO), zinc oxide nanoparticles, and zinc oxide micron particles.

In some embodiments the antimicrobial compound is an additive to a polymeric material from which the probe housing is manufactured.

In some embodiments the antimicrobial compound is dispersed homogeneously throughout the polymeric material from which the probe housing is manufactured.

In some embodiments the antimicrobial compound is dispersed nonhomogeneously throughout the polymeric material from which the probe housing is manufactured, such that a relatively greater amount of the antimicrobial compound is present at the exterior surface of the probe housing compared to an interior of the probe housing.

In some embodiments the polymeric material from which the probe housing is manufactured is selected from polypropylene, polyethylene, polyvinylchloride, polyurethane, polysulfone, polyphenylsulfone, acrylonitrile butadiene styrene, polyethylene terephthalate, polyamide, polyarcrylates, polyvinyl acetates, polyimide, polyamideimide, polymethylmethacrylate, polyetherimide, polyetheretherketone, polyethersulfone, polycarbonate, and polyester.

In some embodiments the polymeric material from which the probe housing is manufactured is selected from polyphenylsulfone, polyurethane, and polypropylene.

In some embodiments the antimicrobial compound is an additive to a polymeric material from which the probe housing is manufactured, and an antimicrobial coating is disposed on the exterior surface of the probe housing, wherein the antimicrobial coating comprises a second antimicrobial compound, which may be the same or different antimicrobial compound.

In some embodiments the second antimicrobial compound is selected from hydrogen peroxide, zinc oxide, ionic zinc (Zn²⁺), chlorhexidine, copper, silver (ionic), silver nanoparticles, triclosan, polyhexamethylene biguanide (PHMB), quaternary ammonium compound, guanidine, guanidinium compounds, porphyrin, methylene blue, toluidine blue, and rose bengal.

In some embodiments the antimicrobial compound is present in an antimicrobial coating disposed on the exterior surface of the probe housing.

In some embodiments the coating is selected from a dip coating, a spray coating, an imbibe coating, and a hydrogel coating.

In some embodiments the antimicrobial activity provides log reductions in gram+ and gram− bacteria after 24 hours in the range from 3 to 7. In some embodiments the antimicrobial activity provides greater than 3 log reductions in gram+ and gram− bacteria after 24 hours. In some embodiments the antimicrobial activity provides greater than 4 log reductions in gram+ and gram− bacteria after 24 hours. In some embodiments the antimicrobial activity provides greater than 5 log reductions in gram+ and gram− bacteria after 24 hours. In some embodiments the antimicrobial activity provides greater than 6 log reductions in gram+ and gram− bacteria after 24 hours. In some embodiments the antimicrobial activity provides greater than 7 log reductions in gram+ and gram− bacteria after 24 hours. Any of the stated antimicrobial activity values can form an upper or lower endpoint of a range.

The antimicrobial compounds can be incorporated in various polymeric probe housing materials including, but not limited to, polyphenylsulfone, polyurethane and polypropylene. The antimicrobial compounds are used in this housing material can be integrated through additives in polymer processing steps. The antimicrobial compound additive can be spread homogeneously or non-homogeneously throughout the whole material. The antimicrobial compound can be bound to the surface in which the mechanism of antimicrobial action is triggered via surface contact.

Antimicrobial compound materials incorporated as a coating or an additive may include, but are not limited to, various chemistries such as hydrogen peroxide, zinc oxide, ionic zinc (Zn²⁺), chlorhexidine, copper, silver (ionic), silver nanoparticles, triclosan, polyhexamethylene biguanide (PHMB), quaternary ammonium compounds, guanidine, guanidinium compounds, porphyrin, methylene blue, toluidine blue, and rose bengal. Hydrogen peroxide can produce reactive oxygen species that target membrane lipids and the DNA of bacteria. Zinc prevents bacteria from uptake of critical metals rendering them susceptible to immune cell attack.

In some embodiments, the antimicrobial compound is dispersed nonhomogeneously throughout the polymeric material from which the probe housing is manufactured, such that a relatively greater amount of the antimicrobial compound is present at the exterior surface compared to an interior of the probe housing. In other words, a concentration of one or more antimicrobial compounds at a surface of the ultrasound probe housing may be higher than a theoretical concentration based on uniform distribution of ingredients of the material from which the housing is fabricated.

It is to be understood that both the foregoing general description and the following detailed description are exemplary and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 is a representation of an ultrasound probe.

FIG. 2 is a partial cross-sectional representation of an ultrasound probe housing.

FIG. 3 is a partial cross-sectional representation of an ultrasound probe housing having a coating.

### DESCRIPTION OF EMBODIMENTS

The disclosure relates to an ultrasound probe with an exterior surface possessing antimicrobial efficacy. The ultrasound probe has a housing which may be impregnated and/or coated with one or more antimicrobial compounds to provide the exterior surface with antimicrobial functionality.

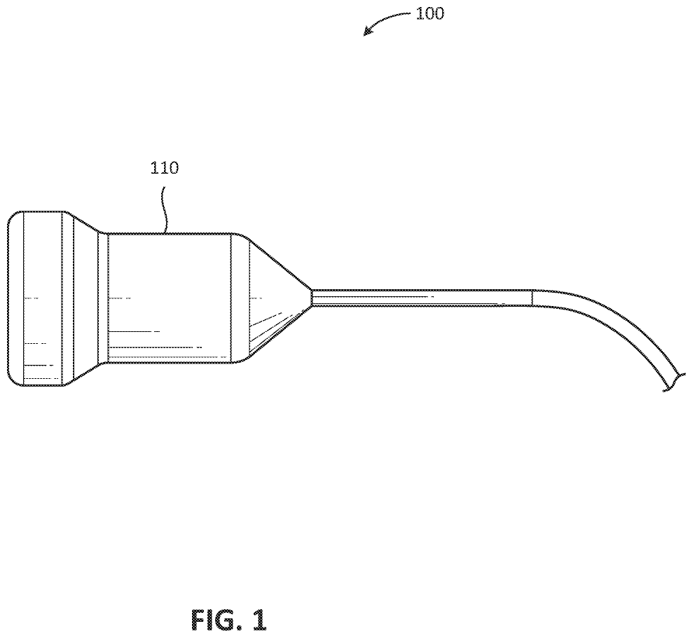

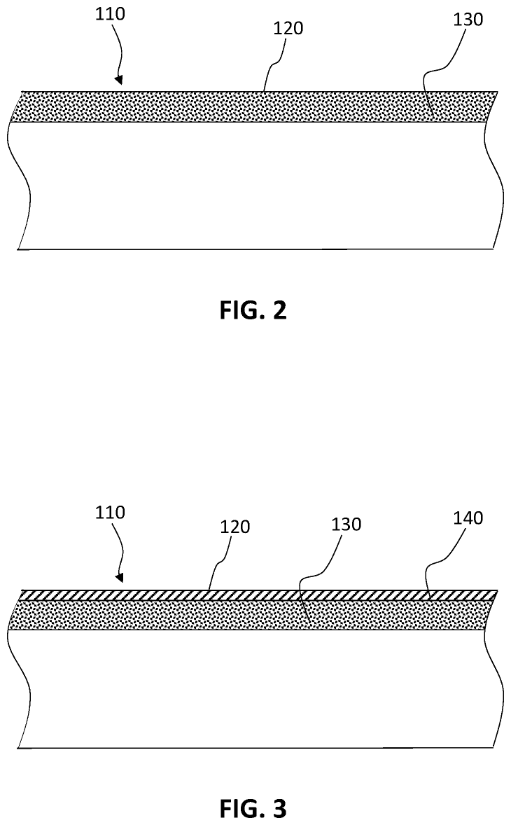

Referring to FIG. 1, in some embodiments an ultrasound probe 100 with an antimicrobial surface activity comprises a housing 110, wherein the housing comprises an antimicrobial compound. FIG. 2 shows a partial cross-sectional representations of the ultrasound probe housing 110 having antimicrobial surface activity. The ultrasound probe 100 has an exterior surface 120. One or more antimicrobial compounds are present at the exterior surface 120 in sufficient concentration to provide antimicrobial activity with microbes that contact the exterior surface 120 of the ultrasound probe 100.

In an embodiment, one or more antimicrobial compound additives are incorporated into the material from which the probe housing is manufactured. Non-limiting examples of suitable antimicrobial compounds include ionic zinc (Zn²⁺), silver nanoparticles, porphyrin, methylene blue, toluidine blue, and rose bengal.

The probe housing may comprise one or more polymeric materials. Probe housing polymeric materials include, but not limited to, polypropylene, polyethylene, polyvinylchloride, polyurethane, polysulfone, polyphenylsulfone, acrylonitrile butadiene styrene, polyethylene terephthalate, polyamide, polyarcrylates, polyvinyl acetates, polyimide, polyamideimide, polymethylmethacrylate, polyetherimide, polyetheretherketone, polyethersulfone, polycarbonate, and polyester.

One or more antimicrobial compounds can be incorporated directly into the ultrasound probe housing material by integrating them directly into the raw resin before molding or by integrating them into a modifier resin which is designed to compatibilize the active antimicrobial compound with the ultrasound probe's housing material.

The antimicrobial compound may be dispersed homogeneously throughout the polymeric material from which the probe housing is manufactured.

The antimicrobial compound may be dispersed nonhomogeneously throughout the polymeric material from which the probe housing is manufactured, such that a relatively greater amount of the antimicrobial compound is present at the exterior surface 120 compared to an interior 130 of the probe housing. In other words, a concentration of one or more antimicrobial compounds at the exterior surface 120 of the ultrasound probe housing 110 may be higher than a theoretical concentration based on uniform distribution of ingredients of the material from which the housing is fabricated.

More than one antimicrobial compound may be dispersed throughout the polymeric material from which the probe housing is manufactured. A plurality of antimicrobial compounds may provide a broader spectrum of antimicrobial activity.

FIG. 3 shows a partial cross-sectional representations of another embodiment of the ultrasound probe housing 110 having antimicrobial surface activity.

One or more antimicrobial compounds may be incorporated into a coating 140 on the ultrasound probe housing 110 such that the one or more antimicrobial compounds are present at the exterior surface 120 of the ultrasound probe in sufficient concentration to provide antimicrobial activity with microbes that contact the exterior surface 120 of the ultrasound probe 100.

Known coating technologies which incorporate the antimicrobial compound may be used. Examples of such coating technologies includes, but are not limited to, a dip coating, a spray coating, an imbibe coating, and a hydrogel coating. In some embodiments a primer chemistry may be used to improve coating adhesion.

Polyurethane is a coating matrix which may be used with dip coating and spray coating technologies. Non-limiting examples of solvents which may be used with polyurethane-based coatings include methanol, ethanol, isopropyl alcohol (IPA), dioxolane, methyl ethyl ketone (MEK), tetrahydrofuran (THF), and acetone. The coating solutions typically dissolve at temperatures in the range of 50° C. to 80° C. The dip coating and spray coating process is typically performed at room temperature. Examples of antimicrobial compounds which may be used in the dip coating and spray coating technologies include, but are not limited to, ionic zinc (Zn²⁺), chlorhexidine, copper, silver (ionic), triclosan, polyhexamethylene biguanide (PHMB), quaternary ammonium compounds, guanidine, and guanidinium compounds.

Imbibe coatings may be prepared by exposing a polymeric substrate to a solvent containing the antimicrobial compound. Typical solvents include, but are not limited to methanol, ethanol, isopropyl alcohol (IPA), dioxolane, methyl ethyl ketone (MEK), tetrahydrofuran (THF), and acetone. The imbibe coating process may be performed at a temperature in the range from room temperature to about 100° C. Examples of antimicrobial compounds which may be used in the imbibe coating include, but are not limited to, chlorhexidine, copper, silver (ionic), triclosan, polyhexamethylene biguanide (PHMB), quaternary ammonium compounds, guanidine, and guanidinium compounds.

A hydrogel is a known coating matrix used with dip coating and spray coating technologies. Non-limiting examples of hydrogels include polyvinyl alcohol, polyethylene glycol (PEG), and polyacrylate-based hydrogels. Non-limiting examples of solvents which may be used with hydrogel-based coatings include water, methanol, ethanol, and isopropyl alcohol (IPA). A pH/acid modifier may be included. The hydrogel coating solutions typically dissolve at temperatures in the range of 60° C. to 95° C. The dip coating and spray coating process is typically performed at room temperature. The hydrogel coating may be cured in formaldehyde. Examples of antimicrobial compounds which may be used in the hydrogel dip coating and spray coating technologies include, but are not limited to, ionic zinc (Zn′), chlorhexidine, copper, silver (ionic), triclosan, polyhexamethylene biguanide (PHMB), quaternary ammonium compounds, guanidine, and guanidinium compounds.

In some embodiments, a combination of one or more antimicrobial compound additives incorporated into the material from which the housing is fabricated, and one or more antimicrobial compounds incorporated into a coating on the surface of the ultrasound probe are utilized to provide self-disinfecting properties.

Regardless of whether the antimicrobial compound is present in a coating or an additive to the probe housing material or a combination of both coating and additive, one or more antimicrobial compounds are present at the exterior surface of the ultrasound probe housing in sufficient concentration to interact with microbes that contact with surface of the probe. This enhanced design covers portions of the ultrasound probe that may contact the patient and clinician. Upon contact, the one or more antimicrobial compounds exert their antimicrobial activity with their respective mechanisms. Furthermore, the antimicrobial compounds will not elute or leach from the device in any capacity. Thus, the disclosed self-disinfecting ultrasound probe will be inexhaustible in providing continuous sterilization of the probe's surface.

Antimicrobial compounds which may be used in disclosed ultrasound probe embodiments to provide antimicrobial activity include, but are not limited to, the antimicrobial compounds listed in Table 1, below.

TABLE 1
Antimicrobial Compounds

| Antimicrobial | | | |
|---|---|---|---|
| Compound | Concentration Ranges | | Concentration In |
| Silver (ionic) | 1-20 | wt. % | Coating on product |
| Silver (ionic) | 1-20 | wt. % | Imbibe solution |
| Chlorhexidine | 1-20 | wt. % | Coating on product |
| Chlorhexidine | 1-20 | wt. % | Imbibe solution |
| PHMB | 1-20 | wt. % | Coating on product |
| PHMB | 1-20 | wt. % | Imbibe solution |
| Ionic zinc (Zn²⁺) | 0.009-6% | | Final product material |
| Zinc oxide + H₂O₂ | 20 wt. % ± 10% | | Coating on product |
| Guanidine | 1-20 | wt. % | Coating on product |
| Guanidine | 1-20 | wt. % | Imbibe solution |
| Guanidinium | 1-20 | wt. % | Coating on product |
| Guanidinium | 1-20 | wt. % | Imbibe solution |
| Triclosan | 1-20 | wt. % | Coating on product |
| Triclosan | 1-20 | wt. % | Imbibe solution |
| Quaternary ammonium | 1-20 | wt. % | Coating on product |
| compound | | | |
| Quaternary ammonium | 1-20 | wt. % | Imbibe solution |
| compound | | | |
| Porphyrin | 0.05-5 | wt. % | Final product material |
| Methylene blue | 0.05-5 | wt. % | Final product material |
| Toluidine blue | 0.05-5 | wt. % | Final product material |
| Rose bengal | 0.05-5 | wt. % | Final product material |
| Silver nanoparticle | 5-50 | ppm | Final product material |

### EMBODIMENTS

Various embodiments are listed below. It will be understood that the embodiments listed below may be combined with all aspects and other embodiments in accordance with the scope of the invention.

Embodiment 1. An ultrasound probe with antimicrobial surface activity comprising a housing, wherein the housing comprises an antimicrobial compound, wherein the antimicrobial compound is present at an exterior surface of the probe housing in sufficient concentration to provide antimicrobial activity with microbes that contact the exterior surface of the ultrasound probe.

Embodiment 2. The ultrasound probe of embodiment 1, wherein the antimicrobial compound is selected from hydrogen peroxide, zinc oxide, ionic zinc (Zn²⁺), chlorhexidine, silver (ionic), silver nanoparticles, triclosan, polyhexamethylene biguanide (PHMB), quaternary ammonium compounds, guanidine, guanidinium, porphyrin, methylene blue, toluidine blue, and rose bengal.

Embodiment 3. The ultrasound probe of any preceding embodiment, wherein the housing comprises a plurality of antimicrobial compounds.

Embodiment 4. The ultrasound probe of any preceding embodiment, wherein the antimicrobial compound is ionic zinc (Zn²⁺).

Embodiment 5. The ultrasound probe of any preceding embodiment, wherein the antimicrobial compound is selected from zinc oxide (ZnO), zinc oxide nanoparticles, and zinc oxide micron particles.

Embodiment 6. The ultrasound probe of any preceding embodiment, wherein the antimicrobial compound is an additive to a polymeric material from which the probe housing is manufactured.

Embodiment 7. The ultrasound probe of embodiment 6, wherein the antimicrobial compound is dispersed homogeneously throughout the polymeric material from which the probe housing is manufactured.

Embodiment 8. The ultrasound probe of embodiment 6, wherein the antimicrobial compound is dispersed nonhomogeneously throughout the polymeric material from which the probe housing is manufactured, such that a relatively greater amount of the antimicrobial compound is present at the exterior surface compared to an interior of the probe housing.

Embodiment 9. The ultrasound probe of any of embodiments 6 through 8, wherein the polymeric material from which the probe housing is manufactured is selected from polypropylene, polyethylene, polyvinylchloride, polyurethane, polysulfone, polyphenylsulfone, acrylonitrile butadiene styrene, polyethylene terephthalate, polyamide, polyarcrylates, polyvinyl acetates, polyimide, polyamideimide, polymethylmethacrylate, polyetherimide, polyetheretherketone, polyethersulfone, polycarbonate, and polyester.

Embodiment 10. The ultrasound probe of any of embodiments 6 through 9, wherein the polymeric material from which the probe housing is manufactured is selected from polyphenylsulfone, polyurethane and polypropylene.

Embodiment 11. The ultrasound probe of any of embodiments 6 through 10, further comprising an antimicrobial coating disposed on the exterior surface of the probe housing, wherein the antimicrobial coating comprises a second antimicrobial compound.

Embodiment 12. The ultrasound probe of embodiment 11, wherein the second antimicrobial compound is selected from ionic zinc (Zn²⁺), chlorhexidine, silver (ionic), copper, triclosan, polyhexamethylene biguanide (PHMB), quaternary ammonium compounds, guanidine, and guanidinium compounds.

Embodiment 13. The ultrasound probe of any of embodiments 11 through 12, wherein the coating is selected from a dip coating, a spray coating, an imbibe coating, and a hydrogel coating.

Embodiment 14. The ultrasound probe of any of embodiments 1 through 5, wherein the antimicrobial compound is present in an antimicrobial coating disposed on the exterior surface of the probe housing.

Embodiment 15. The ultrasound probe according to claim 14, wherein the coating is selected from a dip coating, a spray coating, an imbibe coating, and a hydrogel coating.

Embodiment 16. The ultrasound probe of any preceding embodiment, wherein the antimicrobial activity provides greater than 3 log reductions in gram+ and gram− bacteria after 24 hours.

Embodiment 17. The ultrasound probe of any preceding embodiment, wherein the antimicrobial activity provides greater than 4 log reductions in gram+ and gram− bacteria after 24 hours.

Embodiment 18. The ultrasound probe of any preceding embodiment, wherein the antimicrobial activity provides greater than 5 log reductions in gram+ and gram− bacteria after 24 hours.

Embodiment 19. The ultrasound probe of any preceding embodiment, wherein the antimicrobial activity provides greater than 6 log reductions in gram+ and gram− bacteria after 24 hours.

Embodiment 20. The ultrasound probe of any preceding embodiment, wherein the antimicrobial activity provides greater than 7 log reductions in gram+ and gram− bacteria after 24 hours.

Other features and advantages of the disclosed invention are apparent from the different examples that follow. The examples below illustrate different aspects and embodiments of the present invention and how to make and practice them. The examples do not limit the claimed invention. Although methods and materials similar or equivalent to those described herein can be used in the practice of the present invention, suitable methods and materials are described below. Based on the present disclosure the skilled artisan can identify and employ other components and methodology useful for practicing the present invention.

### EXAMPLES

### Example 1

The antimicrobial surface activity of a polymeric material from which an ultrasound probe housing may be manufactured was tested. The polymeric material contained an antimicrobial compound comprising ionic zinc (Zn²⁺). The antimicrobial surface activity was verified according to one or more of the following standardized tests: ISO 22196, JIS Z 2801, ASTM E2149, ISO 20743, and FZ/T73023. The antimicrobial surface had an efficacy in the range of 3 Log reductions (99.9% reduction) to 7 Log reductions (99.99999%). The antimicrobial surface was effective against Staphylococcus aureus, Staphylococcus epidermidis, Clostridium difficile, Listeria, MRSA, Streptococcus mitis, Streptococcus sanguinis, a hemolytic streptococcus, Escherichia coli, Salmonella, Acinetobacter baumannii, Pseudomonas aeruginosa, Enterobacter cloacae, Legionella Pneumophila, Fusobacterium nucleatum, Eikenella corrodens, and Klebsiella pneumoniae.

### Example 2

The antimicrobial surface activity of a substrate was tested. The substrate was coated with an aqueous dispersion comprising zinc oxide, a polymer, and hydrogen peroxide configured to provide a durable coating on the substrate.

Table 2 reports the antimicrobial surface activity at 24 and 72 hours against biofilms of Candida albicans, Staphylococcus aureus, and Pseudomonas aeruginosa.

TABLE 2

| | 24 hour Biofilm | | 72 hour Biofilm | |
|---|---|---|---|---|
| | Log | | Log | |
| Strains | Reduction | % Kill | Reduction | % Kill |
| Candida albicans | 0.22 | 39.18 | 0.39 | 58.90 |
| Staphylococcus aureus | 7.96 | 100.00 | 7.49 | 100.00 |
| Pseudomonas aeruginosa | 6.21 | 100.00 | 4.52 | 100.00 |

### Example 3

The antimicrobial surface activity of a substrate was tested. The substrate was coated with a coating comprising guanidine or a coating comprising a porphyrin.

Table 3 reports the antimicrobial surface activity at 24 hours against biofilms of Candida albicans, Staphylococcus aureus, and Pseudomonas aeruginosa in two different media.

TABLE 3

| | 24 hour Biofilm | | 24 hour Biofilm | |
|---|---|---|---|---|
| | Guanidine antimicrobial | | Porphyrin antimicrobial | |
| | compound | | compound | |
| | Log | | Log | |
| Strains | Reduction | % Kill | Reduction | % Kill |
| Candida albicans in Media 1 | 5.68 | 100.00 | 5.86 | 100.00 |
| Candida albicans in Media 2 | 5.69 | 100.00 | 5.69 | 100.00 |
| Staphylococcus aureus Media 1 | 7.75 | 100.00 | 7.07 | 100.00 |
| Staphylococcus aureus Media 2 | 2.64 | 99.77 | 2.74 | 99.82 |
| Pseudomonas aeruginosa Media 1 | 7.46 | 100.00 | 7.46 | 100.00 |
| Pseudomonas aeruginosa Media 2 | 3.98 | 99.99 | 3.98 | 99.99 |

### Example 4

The antimicrobial surface activity of a substrate was tested. The substrate was a 2.5 cm×2.5 cm polyurethane square coated with a coating comprising a quaternary ammonium compound.

Table 4 reports the antimicrobial surface activity at 24 and 72 hours against biofilms of Staphylococcus aureus, Klebsiella pneumoniae, Candida albicans, and Staphylococcus epidermidis in different media broths.

TABLE 4

| | | Log | |
|---|---|---|---|
| | | Reduction | |
| | | 24 | 72 |
| Strains | Broths | Hour | Hour |
| S. aureus | 1/500 NB | >5 | >4.5 |
| K. pneumoniae | 1/500 NB | >5 | >5 |
| C. albicans | 1/500 NB | >5 | >5 |
| S. epidermidis | 20% NB in Butterfields phosphate buffer | >5 | >5 |
| K. pneumoniae | 10% NB in Butterfields phosphate buffer | >5 | >5 |
| C. albicans | 20% NB in Butterfields phosphate buffer | >5 | >5 |

Where “NB” is a neutralizing broth.

From the foregoing examples, antimicrobial studies have been performed on ultrasound probe surface materials to assess their antimicrobial efficacy. Greater than 4 log reductions and sometimes as high as 7-8 log reductions were found in gram+ and gram− bacteria at time points of 24 hours and 72 hours for certain ultrasound probe surface materials relative to a control ultrasound probe surface material. This suggests that the disclosed antimicrobial chemistries when integrated to the ultrasound probe will passively disinfect the probe without the requirement of additional workflow steps and capital equipment.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

1. An ultrasound probe with antimicrobial surface activity comprising a housing, wherein the housing comprises an antimicrobial compound, wherein the antimicrobial compound is present at an exterior surface of the probe housing in sufficient concentration to provide antimicrobial activity with microbes that contact the exterior surface of the ultrasound probe.

2. The ultrasound probe according to claim 1, wherein the antimicrobial compound is selected from hydrogen peroxide, zinc oxide, ionic zinc (Zn²⁺), chlorhexidine, silver (ionic), silver nanoparticles, triclosan, polyhexamethylene biguanide (PHMB), quaternary ammonium compounds, guanidine, guanidinium compounds, porphyrin, methylene blue, toluidine blue, and rose bengal.

3. The ultrasound probe according to claim 1, wherein the housing comprises a plurality of antimicrobial compounds.

4. The ultrasound probe according to claim 1, wherein the antimicrobial compound is ionic zinc (Zn²⁺).

5. The ultrasound probe according to claim 1, wherein the antimicrobial compound is selected from zinc oxide (ZnO), zinc oxide nanoparticles, and zinc oxide micron particles.

6. The ultrasound probe according to claim 1, wherein the antimicrobial compound is an additive to a polymeric material from which the probe housing is manufactured.

7. The ultrasound probe according to claim 6, wherein the antimicrobial compound is dispersed homogeneously throughout the polymeric material from which the probe housing is manufactured.

8. The ultrasound probe according to claim 6, wherein the antimicrobial compound is dispersed nonhomogeneously throughout the polymeric material from which the probe housing is manufactured, such that a relatively greater amount of the antimicrobial compound is present at the exterior surface compared to an interior of the probe housing.

9. The ultrasound probe according to claim 6, wherein the polymeric material from which the probe housing is manufactured is selected from polypropylene, polyethylene, polyvinylchloride, polyurethane, polysulfone, polyphenylsulfone, acrylonitrile butadiene styrene, polyethylene terephthalate, polyamide, polyarcrylates, polyvinyl acetates, polyimide, polyamideimide, polymethylmethacrylate, polyetherimide, polyetheretherketone, polyethersulfone, polycarbonate, and polyester.

10. The ultrasound probe according to claim 6, wherein the polymeric material from which the probe housing is manufactured is selected from polyphenylsulfone, polyurethane and polypropylene.

11. The ultrasound probe according to claim 6, further comprising an antimicrobial coating disposed on the exterior surface of the probe housing, wherein the antimicrobial coating comprises a second antimicrobial compound.

12. The ultrasound probe according to claim 11, wherein the second antimicrobial compound is selected from ionic zinc (Zn²⁺), chlorhexidine, copper, silver (ionic), triclosan, polyhexamethylene biguanide (PHMB), quaternary ammonium compounds, guanidine, and guanidinium compounds.

13. The ultrasound probe according to claim 11, wherein the coating is selected from a dip coating, a spray coating, an imbibe coating, and a hydrogel coating.

14. The ultrasound probe according to claim 1, wherein the antimicrobial compound is present in an antimicrobial coating disposed on the exterior surface of the probe housing.

15. The ultrasound probe according to claim 14, wherein the coating is selected from a dip coating, a spray coating, an imbibe coating, and a hydrogel coating.

16. The ultrasound probe according to claim 1, wherein the antimicrobial activity provides greater than 3 log reductions in gram+ and gram− bacteria after 24 hours.

17. The ultrasound probe according to claim 1, wherein the antimicrobial activity provides greater than 4 log reductions in gram+ and gram− bacteria after 24 hours.

18. The ultrasound probe according to claim 1, wherein the antimicrobial activity provides greater than 5 log reductions in gram+ and gram− bacteria after 24 hours.

19. The ultrasound probe according to claim 1, wherein the antimicrobial activity provides greater than 6 log reductions in gram+ and gram− bacteria after 24 hours.

20. The ultrasound probe according to claim 1, wherein the antimicrobial activity provides greater than 7 log reductions in gram+ and gram− bacteria after 24 hours.
